(19) United States
(12) Patent Application Publication
(10) Pub. No.: US 2005/0233324 A1
(43) Pub. Date: Oct. 20, 2005

(54) Device for the amplification of dna, comprising a microwave energy source

(76) Inventors: John Michael Corbett, Sanctuary Cove (AU); John Michael Corbett Jr, Bellevue Hill (AU)

Correspondence Address: STEPTOE & JOHNSON LLP, 1330 CONNECTICUT AVENUE, N.W., WASHINGTON, DC, 20036, US

(21) Appl. No.: 10/512,972

(22) PCT Filed: May 1, 2003
(86) PCT No.: PCT/AU03/00515

§ 371 Date: Jun. 13, 2005

(30) Foreign Application Priority Data
May 1, 2002 (AU) PS 2058

(51) Int. Cl. C12Q 1/68; C12P 19/34; C12M 1/34; H05B 6/64

(52) U.S. Cl. 435/6; 435/91.2; 435/287.2; 219/679

(57) ABSTRACT

The invention relates to a device for the amplification of DNA in a reaction mixture, the device (1) comprising a heated chamber (2) including a rotor (3) for holding a plurality of reaction vessels for reaction mixtures, a drive means for the rotor, a microwave energy source (8) with means for controlled delivery of said energy to the reaction mixtures, and a system (14-16) for determining denaturation of double-stranded DNA. The invention also provides a method for the amplification of a nucleic acid strand. In the first step of the method, a reaction mixture is formed comprising the target nucleic acid strand, nucleotides, a primer, a thermostable nucleic acid polymerase, and, if necessary, a reagent for the detection of denaturation of double-stranded DNA. In the second step, the mixture is incubated at a temperature which allows synthesis of a nucleic acid strand complementary to the target nucleic acid strand. The third step comprises denaturing double-stranded DNA formed in the second step by microwave energisation of the reaction mixture with monitoring of the mixture to determine the denaturation end point. The reaction mixture is allowed to cool to a temperature at which primer anneals to the target nucleic acid strand in the fourth step. The second to fourth steps are repeated until a desired level of amplification is achieved.

### TECHNICAL FIELD

The invention the subject of this application relates to procedures that require the rapid cycling of a reaction mixture between different conditions. More particularly, the invention relates to a device and method for use in such cycling procedures. The device and method are particularly suited for amplifying DNA in processes involving successive cycles of annealing, polymerisation and denaturation steps.

### BACKGROUND ART

In a number of applications such as gene analysis and DNA profiling, it is desirable to multiply the amount of particular nucleic acid sequences present in a sample. For example, a duplex DNA segment of up to approximately six thousand base pairs in length may be amplified many million fold by means of the polymerase chain reaction (PCR), starting from as little as a single copy. In this technique, a denatured duplex DNA sample is incubated with a molar excess of two oligonucleotide primers, one being complementary to a short strand of the DNA duplex and the other being identical to a second short sequence upstream of it (i.e., more 5′).

Each primer anneals to its complementary sequence and primes the template-dependent synthesis by DNA polymerase of a complementary strand which extends beyond the site of annealing of the other primer through the incorporation of deoxynucleotide triphosphates. Each cycle of denaturation, annealing and synthesis affords an approximate doubling of the amount of target sequence, where the target sequence is defined as the DNA sequence subtended by and including the primers. A cycle is controlled by varying the temperature to permit successive denaturation of complementary strands of duplex DNA, annealing of the primers to their complementary sequences, and primed synthesis of new complementary sequences. The use of a thermostable DNA polymerase obviates the necessity of adding new enzyme for each cycle, thus allowing automation of the DNA amplification process by thermal cycling. Twenty amplification cycles increases the amount of target sequence by approximately one million-fold.

More detailed information regarding the polymerase chain reaction can be found in standard texts such as PCR Protocols—A Guide to Methods and Application (M. A. Innis, D. H. Gelfard, J. J. Sainskey and T. J. White ed's, Academic Press, Inc., San Diego, 1990), the entire content of which is incorporated herein by cross reference.

It has been found that the technique of DNA polymerization requires rapid controlled heating and cooling cycles. The art is replete with incubators and other devices to achieve this end—see, for example, International Patent Application No. PCT/AU90/00560 (Publication No. WO 91/07504) and International Patent Application No. PCT/AU98/00277 (Publication No. WO 98/49340).

Typically, a device used for PCR consists of a heat conductive material provided with channels or cavities adapted to receive vessels in which the reaction is to take place, for example Eppendorf™ tubes. The heat conductive material is then provided with heating/cooling means.

Wittwer et al. in Biotechniques 10, 76-82 (1991) state that in commercial units for automated DNA amplification, temperature transition rates are usually no less than 3° C. per second when metal blocks or water are used for thermal equilibration and samples are contained in plastic micro-centrifuge tubes. A significant fraction of cycle time is spent heating and cooling the sample, as opposed to being spent at optimal denaturation, annealing and elongation temperatures. Extended amplification times of two to four hours are common and long transition times make it difficult to determine optimal temperatures and times for each stage. Instantaneous temperature changes are not possible because of sample, container and cycler heat capacities.

In the international application referred to above—PCT/AU98/00277, the entire content of which is incorporated herein by cross reference—there is disclosure of a method and device for thermal cycling of reaction mixtures which at least partially overcome the temperature transition rate problem referred to in the previous paragraph. In the PCT/AU98/00277 device and method, reaction mixture vessels are held in a rotor which rotates in a controlled temperature environment. Temperature cycling is effected by heating and cooling of the environment. This allows more efficient heating and cooling of the reaction mixtures since transition losses are minimised. Also, the fact that the samples are spinning ensures that all samples are heated and cooled at the same rate. Consequently, no equilibration times are required when a set point temperature is reached.

While the PCT/AU98/00277 device and method reduces cycle time, the time is nevertheless too long for use in a linear amplification process. Such a process is desirable as it allows amplification of a single DNA or cDNA strand. Other available amplification devices and methods similarly have cycle times of such a period that use for linear amplification is not feasible.

It would therefore be desirable to have available a device and method suitable for linear amplification of a nucleic acid strand.

### SUMMARY OF THE INVENTION

An object of the invention is to provide a device and method for the cycling of a reaction mixture in which the cycle time is such that it allows the device and method to be used for the linear amplification of DNA.

According to a first embodiment of the invention, there is provided a device for the amplification of DNA in a reaction mixture, the device comprising:
a heated chamber including a rotor for holding a plurality of reaction vessels for reaction mixtures;
a drive means for said rotor;
a microwave energy source with means for controlled delivery of said energy to said reaction mixtures; and
a system for determining denaturation of double-stranded DNA.

According to a second embodiment of the invention, there is provided a method for the amplification of a nucleic acid strand, the method comprising the steps of:
i) forming a reaction mixture comprising said target nucleic acid strand, nucleotides, a primer, a thermostable nucleic acid polymerase, and, if necessary, a reagent for the detection of denaturation of double-stranded DNA;
ii) incubating said mixture at a temperature which allows synthesis of a nucleic acid strand complementary to said target nucleic acid strand;
iii) denaturing double-stranded DNA formed in step (ii) by microwave energisation of said reaction mixture with monitoring of said mixture to determine the denatur-ation end point;
iv) allowing said reaction mixture to cool to a temperature at which primer anneals to said target nucleic acid strand; and
v) repeating steps (ii) to (iv) until a desired level of amplification is achieved.

It will be appreciated from the foregoing definitions of embodiments of the invention that the invention relies on microwave energy to denature DNA rather than heat as in conventional procedures. In this way, faster cycles are possible as there is no need to externally heat reaction mixtures to denature DNA or to delay the primer annealing and polymerisation steps while the mixture cools to the set temperature for primer annealing and polymerisation.

With regard to the first embodiment of the invention, the device is essentially the same as that described in PCT/AU98/00277, but modified to include the microwave energy denaturisation feature. In broad terms, the device chamber can be any suitable, typically insulated, container for the internal device components and for association of ancillary components therewith. The chamber advantageously has a lid or sealable opening for loading the device rotor.

Heating of the chamber can be by any of the means used for controlled heating of automated reaction devices. Typically, heating is by a heater located within the chamber with circulation of heated air within the chamber aided by a fan. Alternatively, heated air can be supplied to the chamber from a port or ports in a chamber wall.

A device according to the invention will normally have a temperature sensor within the chamber which is linked to an associated computer responsible for controlling the operation of the device. Through sensing of the chamber temperature, heater operation can be controlled via the computer.

With regard to the device rotor, this can be any suitable rotor provided that it is not fabricated from a metals material. Rotors are also preferably not heat conductive or electrically conductive. This is to avoid heating of the rotor during microwave energisation of reaction mixtures. Heat accumulation by the rotor would prevent maintenance of the reaction mixture at the annealing and polymerisation temperature. Advantageously therefore, rotors are fabricated from a plastics material. Rotors are typically a flat disc with an annular ring forming an outward portion thereof which is angled upwardly and has apertures therein for holding a plurality of reaction tubes. The rotor can be a disposable item which is used for a single set of amplifications.

The rotor drive means can be any drive means used for rotor devices in scientific equipment. For example, the drive means can be a direct-coupled AC motor, a DC motor, or an AC motor that drives the rotor via a gearbox or pulleys or the like. Preferably, the drive means is a direct-coupled AC motor, DC motor or stepper motor with the motor external to the chamber.

The microwave energy source typically comprises a magnetron that is external to the device chamber with energy delivered to reaction vessels via a wave guide or any other means for delivering microwave energy known to those of skill in the art.

The system for determining denaturation of double-stranded DNA can be any system known to those of skill in the art. Typically, the system is a fluorescence detection system which will be described in greater detail below. However, the system can also by an infrared measurement system comprising a standard commercially-available IR detection element mounted on the side of the device chamber and focused on the tips of the reaction vessels to monitor vessel temperature. If necessary, compensation can be made for differences between the temperature of a reaction mixture at the point of DNA denaturation and the temperature of the vessel wall at that point.

The light source and detector for the fluorescence detection system comprises standard components known to those of skill in the art. For example, the light source can be an LED, a laser light source or a halogen lamp, with an appropriate filter to provide light of an appropriate wavelength for excitation of the fluorophore in the reaction mixture.

Emitted fluorescence is typically filtered and then measured by a photomultiplier tube, CCD array, photodiode or CCD camera. The light source and detector are advantageously linked to the associated computer whereby the application of microwave energy is controlled. It will be appreciated by one of skill in the art that on denaturation of double stranded DNA, there will be a drop in the fluorescence of the reaction mixture at which point application of microwave energy can be terminated. Advantageously, a double-stranded DNA reference standard—for example, genomic DNA or cDNA—of a known concentration can be included in reaction mixtures for the purposes of monitoring when denaturation occurs.

Devices according to the invention can optionally include a mechanism for cooling the device chamber. Such a mechanism typically comprises an air supply to the chamber wherein the air is either at ambient temperature or less than ambient by passage through or over a cooling means.

The fluorescence detector used to monitor denaturation of double stranded DNA can also be used to monitor the progress of a reaction. For example, the level of fluorescence prior to denaturation can be used to assess the amount of DNA synthesised. Devices can further-more include additional monitoring equipment such as a spectrophotometer or photometer. The additional monitoring equipment can be dedicated to assessing the progress of a reaction while the fluorescence detection system acts solely as a denaturation monitor.

As alluded to above, operation of the device at the beginning and end of the amplification, and through each denaturation, annealing and polymerisation cycle is advantageously controlled by an associated computer. The computer can control such operations as:
rotor start up and speed (any speed greater than 100 rpm is suitable but typically rotors are rotated ate 500 rpm);
the annealing and polymerisation temperatures and the time for each of these steps; the period of application of microwave energy during the denaturation step;
processing of data generated by the denaturation system and any system used to measure the amount of DNA synthesised;
rotor braking; and
cooling of the device chamber if necessary such as at the beginning and end of the amplification.

It will be appreciated that the computer can be used to control any other equipment or mechanisms associated with the device.

With regard to the second embodiment of the invention, the method is advantageously carried out using the device according to the first embodiment. It will be appreciated however that it is not essential that the device according to the invention be used for the method which is amenable to adaptation to other devices.

With regard to step (i) of the method, the reaction mixture can be any mixture used for an amplification reaction. Typical reaction mixtures are described, for example in standard reference texts such as PCR: a Practical Approach (M J McPherson et al., Ed's), IRL Press, Oxford, England, 1991, and numerous brochures provided by suppliers of amplification reagents and consumables.

The term “target nucleic acid strand” is used herein in the context of a linear amplification to denote one of the strands of a double stranded DNA molecule (genomic or cDNA) to which the primer will anneal to provide a complementary sequence thereto in the amplification reaction. However, the method can be used for non linear amplification of double stranded DNA in which case the reaction mixture will include a primer for each strand of the DNA molecule. Exponential amplification of the double stranded target will result with repetition of the steps of the method.

With regard to the reagent for the detection of the denaturation of double stranded DNA which can be included in reaction mixtures, this will be a fluorophore required for the optical temperature calibration or fluorescence detection systems referred to above. The intercalating fluorophore can be any of those known to persons skilled in the art and include ethidium bromide and SYBR™ Green.

The annealing and polymerisation steps can be carried out at any of the temperatures and for the times used in known amplification procedures.

In current instruments for performing PCR and other linear and non-linear DNA amplification reactions, energy is supplied to the reaction mixture via the reaction vessel wall (glass or plastic) which acts as an insulator. This process usually takes 1 to 2 minutes for a single cycle (55° C.-95° C.-55° C.). In a typical reaction, 30 to 50 cycles are required.

The device according to the invention allows a reaction vessel to be held with its wall at typically 50-65° C. with energy for the denaturation being supplied to the reaction mixture by energizing the magnetron (typically for several seconds). As the reaction vessel wall is held at the typical annealing temperature of 50-65° C., the mixture returns to that temperature shortly after application of microwave energy is terminated due to the fact that the reaction vessel does not absorb an appreciable amount of microwave energy. This reduces the cycle time to 6 to 10 seconds.

Because of the fast cycle time possible with the device and method of the invention, a PCR amplification of double stranded DNA can be performed in minutes rather than hours. More importantly, linear reactions for the amplification of a single strand of DNA can be executed in 1 to 2 hours. This is not achievable with existing devices and methods which require 100 to 1,000 cycles taking up to 30 hours. The device and method of the invention thus allow real time assays to be done at least an order of magnitude faster than currently-available assays.

Having broadly described the invention, a device will now be exemplified with reference to the accompanying drawing briefly described hereafter.

### BRIEF DESCRIPTION OF THE DRAWING

FIG. 1 is a schematic cross section of a device according to the invention.

### DETAILED DESCRIPTION OF A PREFERRED EMBODIMENT

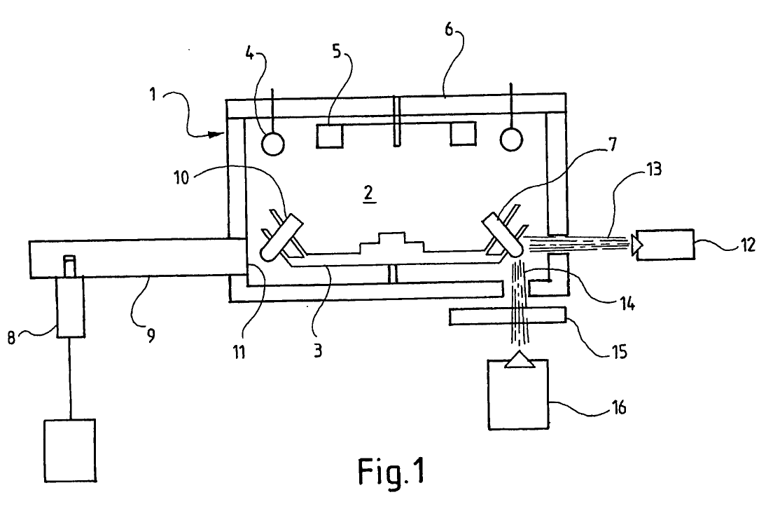

With reference to FIG. 1, device 1 comprises a chamber 2 having rotor 3 which is driven by a stepper motor not shown in the drawing. Chamber 2 also includes a radial heater 4 and a fan 5 for distributing heated air throughout the chamber. Heater 4 and fan 5 are mounted to hinged lid 6 of the device, which lid can be pivoted out of the way to gain access to rotor 3. Rotor 3 has a plurality of holes for holding reaction vessels, one of which vessels is item 7.

Device 1 also includes a magnetron 8 from which microwaves can be directed via wave-guide 9 to reaction vessels such as 10 as they pass the microwave emission point 11. A light source 12 is provided for illuminating a reaction vessel as it passes through beam 13. Light 14 emitted from reaction vessel 7 passes through filter 15 to be detected by photomultiplier tube 16.

Device components such as the rotor drive, heater 4, fan 5, magnetron 8, and light source 12, are controlled by a computer not shown in the drawing.

Operation of the device is as follows. Reaction mixtures are dispensed into reaction vessels using manual pipettors or automated robotic pippetting means and heated to the annealing temperature via heater 4 and fan 5 under the control of the associated computer. Rotor 3 is rotated at greater than 100 rpm under the control of the computer during this step and subsequent steps to average reaction vessel temperatures.

On command from the computer to denature double stranded DNA formed in the reaction mixture, magnetron 8 is activated and microwave energy transferred to reaction mixtures via wave guide 9. At least one reaction mixture contains an intercalating dye such as ethidium bromide or SYBR™ Green. The dye is excited by light source 12 and fluorescence measured by photomultiplier tube 16 after selection of light of the appropriate wavelengths by filter 15.

With denaturation of the double stranded DNA, fluorescence emission diminishes and on reaching a present level causes the computer to deactivate magnetron 8.

On termination of application of microwave energy, the reaction mixture quickly returns to the annealing temperature maintained within chamber 2 by heater 3 and fan 4. The cooling of the annealing temperature takes only seconds as the reaction vessel per se is not heated by the microwave energy—only the reaction mixture is acted on by that energy.

At the annealing temperature, the progress of the reaction can be monitored by way of a fluorescent probe present in reaction mixtures or by measuring the increased energy of the intercalating referred to above dye. This monitoring is by way of light source 12, filter 15 and photomultiplier tube 16. Results can be recorded by the computer.

It will be appreciated by a person of skill in the art that many changes can be made to the device and its use as exemplified above without departing from the broad ambit and scope of the invention.

The term “comprise” and variants of the term such as “comprises” or “comprising” are used herein to denote the inclusion of a stated integer or stated integers but not to exclude any other integer or any other integers, unless in the context or usage an exclusive interpretation of the term is required.

The reference to the publications cited in the Background Art section of this specification is not an admission that the disclosures constitute common general knowledge in Australia.

1. A device for the amplification of DNA in a reaction mixture, the device comprising:
a heated chamber including a rotor for holding a plurality of reaction vessels for reaction mixtures;
a drive means for said rotor;
a microwave energy source with means for controlled delivery of said energy to said reaction mixtures; and
a system for determining denaturation of double-stranded DNA.

2. The device of claim 1, wherein said chamber has a lid or sealable opening for loading the device rotor.

3. The device of claim 1, wherein said heating of said chamber is by an internal heater or by supplying heated air through at least one port in a wall of said chamber.

4. The device of claim 1, which further includes temperature sensor within the chamber which is linked to an associated computer responsible for controlling the operation of the device.

5. The device of claim 1, wherein said rotor is fabricated from a plastics material.

6. The device of claim 1, wherein said rotor drive means is a direct-coupled AC motor, DC motor or stepper motor.

7. The device of claim 1, wherein said microwave energy source comprises a magnetron that is external to the device chamber with energy delivered to reaction vessels via a wave guide.

8. The device of claim 1, wherein said system for determining denaturation of double-stranded DNA is a fluorescence detection system or an infrared measurement system.

9. The device of claim 1, which further includes a mechanism for cooling the device chamber.

10. A method for the amplification of a nucleic acid strand, the method comprising the steps of:
i) forming a reaction mixture comprising said target nucleic acid strand, nucleotides, a primer, a thermostable nucleic acid polymerase, and, if necessary, a reagent for the detection of denaturation of double-stranded DNA;
ii) incubating said mixture at a temperature which allows synthesis of a nucleic acid strand complementary to said target nucleic acid strand;
iii) denaturing double-stranded DNA formed in step (ii) by microwave energisation of said reaction mixture with monitoring of said mixture to determine the denaturation end point;
iv) allowing said reaction mixture to cool to a temperature at which primer anneals to said target nucleic acid strand; and
v) repeating steps (ii) to (iv) until a desired level of amplification is achieved.

11. The method of claim 10, wherein said method is carried out in a device according to claim 1.

12. The method of claim 10, wherein said reagent for the detection of the denaturation of double stranded DNA is a fluorophore.
